(19) United States
(12) Patent Application Publication
(10) Pub. No.: US 2016/0159922 A1
(43) Pub. Date: Jun. 9, 2016

(54) Novel Antibodies for the Diagnosis and Treatment of Rheumatoid Arthritis

(71) Applicant: Vilara AB, Lidingo (SE)

(72) Inventors: Lars KLARESKOG, Stockholm (SE); Vivianne MALMSTRÖM, Stockholm (SE); Johanna Maria ÅKERBLÖM STEEN, Stockholm (SE); Daniel John LIGHTWOOD, Berkshire (GB); Stephen Edward RAPECKI, Berkshire (GB)

(21) Appl. No.: 14/906,854

(22) PCT Filed: Jul. 24, 2014
(86) PCT No.: PCT/EP2014/002023

§ 371 Date: Jan. 21, 2016

(30) Foreign Application Priority Data
Jul. 24, 2013 (SE) 1300501-2

(51) Int. Cl. C07K 16/36; C07K 16/18

(52) U.S. Cl. CPC C07K 16/36; C07K 16/18; C07K 2317/565; C07K 2317/21; C07K 2317/34; C07K 2317/33

(57) ABSTRACT

Novel antibodies or binding fragments thereof, which exhibit specific binding to citrullinated epitopes are disclosed, as well as pharmaceutical compositions comprising said antibodies, use of the antibodies and compositions comprising the same, in diagnosis, in prognostics, treatment and/or alleviation of rheumatoid arthritis, as well as processes for generating and preparing said antibodies.

### TECHNICAL FIELD

The present invention relates to novel antibodies as such, and their use in therapy and diagnosis, in particular in the therapy and diagnosis of rheumatoid arthritis, and their utility as a research tool in the study of rheumatoid arthritis.

### BACKGROUND ART

Rheumatoid arthritis is a heterogeneous and partially genetically determined inflammatory disease, where autoimmunity has been assumed to play an important pathogenic role, but where the specificity of the autoimmune reactions and the genetic determinants of these reactions remain incompletely understood.

Therapies of rheumatoid arthritis and other inflammatory and autoimmune diseases have so far been based on manipulation of immune and inflammatory events without knowing the detailed genetic and immunological basis of the disease. These therapies include traditional Disease-Modifying Anti-Rheumatic therapies (DMARD:s), including the most commonly used drug methotrexate, as well as new “biological” therapies that affect cytokine regulation or broad aspects of T and B cell activation and migration. Also, many novel therapies are developed that are not based on detailed knowledge of the genetics and specificity of the autoimmune reactions in rheumatoid arthritis, but which affect general signaling pathways. Altogether, the available treatments of rheumatoid arthritis are insufficient and have side effects. Thus, there is a need for improved treatments of rheumatoid arthritis.

Autoimmune reactions to certain epitopes of self-antigens most likely contribute to the development of rheumatoid arthritis. Antibodies against the patient's own proteins, in particular against collagen type II, alpha-enolase, vimentin, fibrinogen and histories have been identified in patients with rheumatoid arthritis.

These antibodies are often—but not always—directed towards citrullinated variants of the proteins. Citrulline is an unconventional amino acid that results from the delmination (or citrullination) of arginine. Citrulilnation is the process by which an arginine residue in a protein is converted to citrulline.

In today's medical practice the diagnosis of immunity in rheumatoid arthritis is limited to the analysis of the presence or absence of autoantibodies towards generic citrullinated proteins using the so called CCP assay, where CCP denotes cyclic citrullinated peptide. The CCP assay (disclosed for example in WO 2003/050542) comprises the detection of antibodies against a mixture of peptides that have not been demonstrated to occur as natural autoantigens (targets of B-cells and antibody secreting cells) in patients with rheumatoid arthritis.

However, diagnostic procedures that detect antibodies to different “self” antigens (these antibodies are called “autoantibodies”) have been described for rheumatoid arthritis, and Include assays for antibodies against certain distinct citrullinated peptides (for example enolase, fibrinogen, collagen II and histone peptides) as well as antibodies against other (non-citrullinated) proteins and peptides, including collagen type II and snRNP (including so called RA33 antigens). For example WO 2008/090360 and Lundberg et al (Arthritis & Rheumatism, Vol. 58, No 10, 3009-3019) describe the identification of citrullinated epitopes from alpha-enolase associated with rheumatoid arthritis (CEP-1). WO 1999/028344 suggests the use of an anti-vimentin antibody for the preparation of a therapeutic substance or of a diagnostic tool for rheumatoid arthritis. Verpoort et al. (Arthritis & Rheumatism, Vol. 56, No 12, pp 3949-3952) discuss the presence of autoreactivity against citrullinated vimentin and fibrinogen.

Tests for the detection of antibodies against citrullinated forms of enolase, vimentin and/or fibrinogen peptides can be used for diagnosis of rheumatoid arthritis. Such tests show the presence or absence of antibodies in patient serum that react with citrullinated epitopes of such peptides as from alpha-enolase, vimentin, fibrinogen, collagen type II, and histones. There is also a need for positive control reagents that binds to the citrullinated peptides used in the antibody detection systems and that can be used to ensure that the analysis works as intended. Sometimes sera from an individual patient (or a pool of patients) with rheumatoid arthritis are used as positive control. However, the supply of such patient sera is limited. Thus, there is a need for reagents that can be used as positive controls in immunological diagnosis of rheumatoid arthritis and that can be produced in large amounts.

Also, there is a need to map the epitopes responsible for inducing various symptoms of rheumatoid arthritis. This is difficult when the epitopes are dependent on the three-dimensional structure of the protein antigen. When the epitopes are non-linear, antibodies can be used for defining distinct epitopes on native proteins. This can be carried out by allowing labeled (e.g. biotinylated) antibodies with known binding specificity to compete with binding of patient sera to citrullinated proteins. This is a known method for defining the reactivity of antibodies of patient sera as reactivity against conformational epitopes which are not present in shorter peptides. However, it is then necessary to use antibodies that react with the epitopes in the same manner as the disease-causing antibodies and which can be produced in large amounts.

Thus, there is a need for novel antibodies that are known to react with satisfactory specificity and affinity with rheumatoid arthritis-associated epitopes in humans, in particular citrullinated epitopes.

### SUMMARY

One object of the present invention is to provide novel, specific antibodies that can be used in the treatment of rheumatoid arthritis.

Another object of the present invention is to provide novel diagnostic tools and research tools for rheumatoid arthritis.

The inventors have produced human recombinant antibodies from patients with rheumatoid arthritis that react with epitopes that may be responsible for inducing rheumatoid arthritis. So far, these antibodies have been shown to react with peptides from alpha-enolase, vimentin and fibrinogen, but they may react also with other autoantigens in their native shape or after modification, for example citrullination. The peptides are shown in Table 6.

One advantage of these antibodies is that they can be produced in large amounts. Another advantage is that they have been generated from antibody secreting cells from patients with rheumatoid arthritis and that they thus have the same reactivity as potentially pathogenic antibodies. Circulating antibodies are mainly produced by plasma cells. These cells are large B cells that have been exposed to antigen and produce and secrete large amounts of antibodies, providing protective immunity. Plasma B cells, as compared to recently activated B cells, are more differentiated and are therefore likely to produce antibodies with high affinity and specificity. Another advantage is that the binding sequences of the inventive antibodies can be identical to those of the pathogenic antibodies in vivo in patients.

These antibodies can be used as positive controls in diagnostic kits for testing for auto-antibodies against citrullinated epitopes in rheumatoid arthritis. The antibodies can also be used for mapping citrullinated epitopes of antibodies from patients.

These antibodies can also be used to investigate which epitope specificity and which other features are sufficient and necessary to induce different symptoms related to rheumatoid arthritis upon transfer to experimental animals. This is especially true since the antibodies have been generated based on Information generated after identifying actual disease-causing antibodies in synovial fluid of patients.

The antibodies are specific for citrullinated versions of proteins and can be used for investigating the degree of citrullination of proteins in a patient or an animal.

Importantly, because the novel antibodies may bind to the same epitope as the disease-causing antibodies they can be used in the treatment and/or alleviation of rheumatoid arthritis. Dominant negative variants of the antibodies will, when administered in sufficient amounts, compete out the disease-causing antibodies, thereby blocking the pathological inflammation.

In a first aspect it is provided an antibody that binds at least one citrullinated peptide, said antibody comprising a heavy chain CDR1 (HCDR1), a heavy chain CDR2 (HCDR2), and a heavy chain CDR3 (HCDR3) selected from the following combinations of sequences (Table 1a) or a substantially identical antibody.

TABLE 1a
Amino acid sequences of antibody complementarity determining regions

Combination No.

| | 1 | 2 | 3 | 4 |
|---|---|---|---|---|
| HCDR1, SEQ ID No. | 19 | 25 | 31 | 71 |
| HCDR2, SEQ ID No. | 20 | 26 | 32 | 79 |
| HCDR3, SEQ ID No. | 21 | 27 | 33 | 88 |

In another aspect it is provided an antibody that binds at least one citrullinated peptide, said antibody comprising a heavy chain CDR1 (HCDR1), a light chain CDR1 (LCDR1), a heavy chain CDR2 (HCDR2), a light chain CDR2 (LCDR2), a heavy chain CDR3 (HCDR3) and a light chain CDR3 (LCDR3) selected from the following combinations of sequences (Table b) or a substantially identical antibody.

TABLE 1b
Amino acid sequences of antibody complementarity determining regions

| | Combination No. | | | |
|---|---|---|---|---|
| | 1 | 2 | 3 | 4 |
| HCDR1, SEQ ID No. | 19 | 25 | 31 | 71 |
| LCDR1, SEQ ID No. | 1 | 7 | 13 | 70 |
| HCDR2, SEQ ID No. | 20 | 26 | 32 | 79 |
| LCDR2, SEQ ID No. | 2 | 8 | 14 | 78 |
| HCDR3, SEQ ID No. | 21 | 27 | 33 | 88 |
| LCDR3, SEQ ID No. | 3 | 9 | 15 | 87 |
| Suggested name of | ACPA1 | ACPA10 | ACPA11 | ACPA42 |
| antibody: | | | | |

In one embodiment, the antibody is an antibody that binds to at least one citrullinated epitope selected from the group consisting of cit-vim (SEQ ID NO 38), and cit-fib (SEQ ID NO 39) and where the combination of CDRs is selected from the group consisting of CDR combinations 1-3. Interestingly, the antibodies do not bind to citrullinated human enolase peptide 1, CEP-1 (SEQ ID NO 37).

The various antibodies described herein form separate embodiments of the invention as described below.

The antibody may comprise at least one human constant region, for example the constant regions of human IgG, preferably a constant region of human IgG1 or IgG4.

Another aspect is a nucleic acid encoding an antibody according to the disclosure herein. The nucleic acid sequences have been determined by the present inventors and are disclosed in the attached sequence listing, incorporated herein by reference.

Another aspect is an antibody according to the invention for use in the treatment of rheumatoid arthritis. Preferably, the antibody is a dominant negative antibody.

Yet another aspect is a method of treating rheumatoid arthritis comprising administrating to a patient in need thereof a therapeutically effective amount of an antibody according to the disclosure herein, for example a dominant negative antibody.

Yet another aspect is a diagnostic kit or a prognostic kit comprising an antibody according to the disclosure herein and an antibody for use in diagnosis or prognostics, for example in diagnosis of rheumatoid arthritis. The term diagnosis here also includes the prediction of risk of developing rheumatoid arthritis and/or diagnosis of rheumatoid arthritis at an early stage, before the onset of clinical symptoms. Without wishing to be bound to a specific theory, it is contemplated that a patient exhibiting antibodies against citrullinated epitopes already at an early stage of rheumatoid arthritis, or even before the onset of clinical symptoms, runs a risk or is predisposed for a more aggressive form of rheumatoid arthritis. Similarly, by following the appearance and amount of antibodies against citrullinated epitopes, the progression of the disease can be monitored.

The assembly of diagnostic and/or prognostic kits, once the relevant antibodies are available, can be done by a skilled person relying on common general knowledge in the art.

### BRIEF DESCRIPTION OF THE FIGURES

The invention will be described below, in the description and examples, with reference to the figures in which:

FIG. 1 shows the binding of anti-citrullinated antibodies to immobilized CCP-1, fibrinogen, enolase-1 or vimentin arginine and citruline containing peptides.

### DETAILED DESCRIPTION

### Definitions

Wild-type antibodies are typically composed of two identical pairs of polypeptide chains, each pair having one light chain and one heavy chain. Each of the heavy and light chains is made up of two distinct regions, referred to as the variable and constant region. Thus there is the variable heavy chain (VH), the constant heavy chain (CH), the variable light chain (VL) and the constant light chain (CL). The variable regions (VH and VL) of an antibody contain the antigen binding sequences of the molecule and thus determine the specificity of an antibody for its target antigen. In the variable region, three loops for each of the variable domains of the heavy chain and light chain forms the antigen-binding site. Each of the three loops is referred to as a complementary-determining region, or “CDR”. There are six CDR:s, three per heavy chain and three per light chain, designated VHCDR1, VHCDR2, VHCDR3, VLCDR1, VLCDR2 and VLCDR3. The variable region outside, and in between, the CDRs is referred to as the framework region (FR).

The term “antibody” as used herein refers to immunoglobulin molecules and immunologically active portions of immunoglobulin molecules, i.e. molecules that contain an antigen binding site that specifically binds an antigen, whether natural or partially or wholly synthetically produced. The term also covers any polypeptide or protein having a binding domain which is, or is homologous to, an immunoglobin molecule. Examples of antibodies are the immunoglobulin isotypes (e.g. IgG, IgE, IgM, IgD and IgA) and their isotypic subclasses (such as for IgG: IgG1, IgG2, IgG3 and IgG4 in humans and IgG1, IgG2a, IgG2b and IgG3 in mice), fragments which comprise an antigen binding domain such as Fab (consisting of VL, VH, CL and CH1 domains), single chain variable fragments (scFv), consisting of the two VH and VL domains linked together by a flexible peptide linker, Fv fragments consisting of the two variable antibody domains VH and VL, dAb fragments consisting of a single variable region and so called diabodies (multivalent or multi-specific fragments constructed by gene fusion and that bind to two (or more) different antigens). The antibody fragments may be modified. For example, the molecules may be stabilized by the incorporation of disulphide bridges. The term also covers multi-specific antibodies, such as bi-specific and tri-specific antibodies.

The antibodies and the fragments or derivatives thereof may be poly- and/or mono-specific. Antibodies comprise monoclonal and polyclonal antibodies of any origin, including murine, rabbit, human and other antibodies, as well as chimeric antibodies comprising sequences from different species, such as partly humanized antibodies, e.g., partly humanized mouse antibodies. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, and any transferred donor framework residues, is encoded by a polynucleotide of human origin or is identical to such an antibody except within its CDRs. The creation of such antibodies is described in, e.g., WO 92/11018 and Jones, 1986, Nature 321. The humanized antibody will optimally also comprise at least a portion of an immunoglobulin constant region, typically that of a human immunoglobulin, and thus will typically comprise a human Fc region. A variety of techniques and methods for humanizing and reshaping non-human antibodies are well known in the art (See Tsurushita & Vasquez, 2004, Humanization of Monocional Antibodies, Molecular Biology of B Cells, 533-545, Elsevier Science (USA), and references cited therein).

It is possible to use recombinant DNA technology to modify an antibody while maintaining the specificity of the antibody. When applied to the present disclosure, such techniques may involve combining the CDRs presented herein with the constant regions plus framework regions obtained from a different immunoglobin molecule. Such techniques may also involve change of carbohydrate structure of the Fab as well as of the Fc parts of the antibodies. Alternatively, or in combination therewith, modified antibodies can be produced through modification of the protein backbone of the antibodies, for example by changing the isotype from IgG1 to other isotypes, including IgG4, and by producing fragments of the antibodies, for example into Fab fragments. In addition Fc engineering approaches may be used such as those described by Presta 2008, Current Opinion in Immunology, 20, 460-470. Such changes may alter the functions of the antibodies, and make them more suitable as therapeutic agents. The term “antibody” as used herein therefore also refers to recombinant and otherwise modified antibodies.

Thus it is possible to produce an antibody as disclosed herein by replacing the CDR regions of an immunoglobin molecule, such as an antibody, with the CDR regions disclosed herein, for example by using recombinant DNA technology.

As antibodies can be modified in a number of ways, the term “antibody” should be construed as covering any immunoglobin molecule or part thereof capable of carrying the Inventive combinations of CDRs in a manner that enables the binding of the combination of CDRs to their epitopes. Thus, this term covers antibody fragments, derivatives, functional equivalent, affinity reagents and homologues of antibodies, humanized antibodies, including any polypeptide comprising an immunoglobulin molecule or an immunologically active portion of an immunoglobulin molecule whether natural or wholly or partly synthetic. Chimeric molecules comprising an Immunoglobulin binding domain, or equivalent, fused to another polypeptide are therefore included. Also included are chimeric antibodies such that the constant regions may be from non-human origin, such as murine origin.

It has been shown that fragments of a whole antibody can bind antigens to the same extent as the whole antibody. Examples of binding fragments include:

1) the Fab fragment consisting of the VI, VH, CL and CH1 domains; 2) F(ab′)2 fragments, a bivalent fragment comprising two linked Fab fragments and 3) single chain Fv molecules (scFv). These are examples of types of antibody fragments that fall within the definition of “antibody” as used in the context of this disclosure.

For certain purposes, it is possible to use antibodies that lack parts of the constant domain as long as they comprise minimally-binding domains comprising the CDRs and necessary scaffold. Thus it is possible to use the Fab-fragments, scFv and other fragments that are described above in diagnostic methods and as research tools. Here it is described how the CDRs are incorporated into scaffolds comprising the constant regions of human IgG1 and also mouse IgG2a. However, other immunoglobin molecules may be used as scaffold for carrying the CDRs according to the disclosure herein. For example, the CDR may be incorporated into an murine antibody by replacing the existing CDRs in the murine antibody with the CDR:s disclosed herein.

The term “specific” is generally used to refer to the situation in which one member of a binding pair will not show any significant binding to molecules other than its specific binding partner (s) and e.g. has less than about 30%, preferably 20%, 10%, or 1% cross-reactivity with any other molecule other than those specified herein. Alternatively, the binding specificity can be expressed as an at least 100-fold binding preference, or at least 1000-fold preference, or at least 10.000-fold or higher binding preference. The antibodies disclosed herein may be multivalent such that they bind specifically to more than one epitope selected from the group consisting of cit-vim and cit-fib as defined in Table 6.

“Isolated” refers to the state in which antibodies, nucleic acids encoding such antibodies and host cells described herein will preferably be. With respect to antibodies and nucleic acid, “isolated” means that antibodies and nucleic acids will generally be free, or substantially free, of material with which they are naturally associated such as other polypeptides or nucleic acids with which they are found in their natural environment or in the environment in which they are prepared, (e.g. cell culture) for example when such preparations is by recombinant DNA technology. When applied to host cells, “isolated” refers to host cells isolated from the organism from where they originate, such as, for example, cells in cell culture. Antibodies, nucleic acids and host cells may be formulated with diluents or adjuvant and still for practical purposes be isolated.

“Amino acid modification” refers to amino acid residue substitutions, insertions and deletions in a polypeptide sequence. “Substitution” refers to the replacement of an amino acid residue at a particular position in a polypeptide sequence with another amino acid residue. “Insertion” refers to the addition of an amino acid residue between two preexisting amino acid residues a particular position in a polypeptide sequence. “Deletion” refers to removal of an amino acid residue at a particular position in a polypeptide sequence.

### DETAILED DESCRIPTION

According to a first aspect it is provided antibodies ACPA1, ACPA10, ACPA11 and ACPA42 with sequences of heavy chain and light chains CDR1, CDR2 and CDR3 according to KABAT system as defined in Tables 2-4.

TABLE 2
CDR1 sequences

| | Heavy Chain | SEQ ID | Light Chain | SEQ ID |
|---|---|---|---|---|
| Antibody | CDR1 sequence | NO | CDR1 Sequence | NO |
| ACPA1 | GYFIH | 19 | RASEDIGSLLA | 1 |
| ACPA10 | DTTYYWG | 25 | GGDDIENQNVN | 7 |
| ACPA11 | RNDKFVVG | 31 | SGNTSNIGYNIVN | 13 |
| ACPA42 | NDLSYWG | 71 | SGSNSNIESNLVS | 70 |

TABLE 3
CDR2 sequences

| | Heavy Chain | SEQ ID | Light Chain | SEQ ID |
|---|---|---|---|---|
| Antibody | CDR2 sequence | NO | CDR2 Sequence | NO |
| ACPA1 | WIHPTGDTKYAQKFQG | 20 | KASSLES | 2 |
| ACPA10 | SIYYRGNTHYNSSLRS | 26 | FDTRRPS | 8 |
| ACPA11 | SINPLGRTFYTTSLKD | 32 | DNDKRPS | 14 |
| ACPA42 | SVHYSGRTYYSPSLRS | 79 | DNNKRFS | 78 |

TABLE 4
CDR3 sequences

| | Heavy Chain | SEQ ID | Light Chain | SEQ ID |
|---|---|---|---|---|
| Antibody | CDR3 sequence | NO | CDR3 Sequence | NO |
| ACPA1 | TNFSPFRH | 21 | QQYNGPSFT | 3 |
| ACPA10 | LDPFDY | 27 | QVYDRKTDHQV | 9 |
| ACPA11 | DGEGVLFDE | 33 | AVWDDDLSGW | 15 |
| ACPA42 | LGCSGGGCYFDFDYW | 88 | GTWDSSLSAGLF | 87 |

Preferably the antibody binds specifically to at least one citrullinated epitope, preferably at least one epitope selected from the group consisting of cit-vim and cit-fib, more preferably at least one citrullinated epitope selected from the group consisting of citrullinated human vimentin defined by residues 60-75 (SEQ ID NO 38) (cit-vim), and citrullinated human fibrinogen defined by residues 36-52 (SEQ ID NO 39) (cit-fib). The sequences of these peptides are shown in Table 6. Interestingly said antibody does not bind to citrullinated human enolase peptide 1 (SEQ ID NO 37) (CEP-1).

Citrulline is an unconventional amino acid that results from posttranslational modification of arginine (deimination of arginine by peptidylarginine deiminases). Citrullination is the process by which an arginine residue in a protein is converted to citrulline. No tRNA exists for citrulline, its presence in proteins is exclusively dependent on posttranslational (enzymatic) modification.

Herein are also disclosed antibodies with CDR sequences that are substantially identical to the disclosed CDR sequences as long as they have the capacity to bind the citrullinated peptides as disclosed herein. Thus, an antibody with CDR sequences with from 1 to 20, from 1 to 10, from 1 to 8, from 1 to 5, from 1 to 4, from 1 to 3, from 1 or 2 or only 1 amino acid modification(s) compared to the disclosed sequences that bind to at least one citrullinated peptide is comprised within the disclosure.

“Substantially identical” refers to sequences and antibodies with less than from 1 to 20, from 1 to 10, from 1 to 8, from 1 to 5, from 1 to 4, from 1 to 3, from 1 or 2, or only 1 amino acid modification(s) compared to the disclosed combination of CDR sequences. For the avoidance of doubt, it should be noted that the number of modifications is counted over the entire sequence of the CDRs, thus the combination of the HCDR1, LCDR1, HCDR2, LCDR2, HCDR3 and LCDR3 sequences.

Preferably the binding of the antibodies to their respective antigens is specific meaning that no (or significantly less) reaction can be measured towards the unmodified (arginine-containing) epitope. The difference in binding is preferably at least a factor of 100, more preferably at least 1000, and most preferably more than 10.000 fold.

The antibody is preferably an antibody that binds to a known antigen. For example least one antibody selected from the group consisting of ACPA1, ACPA10, ACPA11 and ACPA42 (Sequence combinations No:s.1-4, Tables 1a or 1b). All of these antibodies bind to one or more of the antigens cit-vim and cit-fib as shown in Table 7.

It may be desirable that the antibodies exhibit multiple reactivity, such that they react with more than one antigen. For example, such an antibody can be used as a positive control for more than one type of diagnostic test.

In some instances it is desirable that the antibodies have high specificity and no or low multiple reactivity. For example an antibody selected from the group consisting of ACPA10, ACPA11 and ACPA42 is useful as a therapeutic antibody and contemplated to have utility in epitope mapping, and as research reagents (immunohistochemical staining of biopsy specimen etc.). For therapeutic applications, a high affinity and high selectivity helps to attain better efficacy and is likely to exhibit fewer or no side effects. For diagnostic applications, the same properties make the antibody more sensitive and thus more useful for diagnosis based on a single reactivity or the exact epitope to a protein.

The antibody is preferably an antibody that binds specifically only to cit-fib, such as antibody ACPA11 and ACPA42 (combination 3 and 4 in Table 1a or Table 1b respectively).

The antibody is preferably an antibody that binds specifically only to cit-vim such as antibody ACPA10 (combination 2, in Table 1a and Table 1b respectively).

The antibody is preferably an antibody that binds more than one peptide, such as antibody ACPA1, which has been shown to bind to both cit-fib and cit-vim, but with substantially higher specificity for cit-vim (combination 1 in Table 1a and Table 1b respectively).

The antibody preferably binds to its target epitope with a high affinity (low KD value). The affinity is preferably in, or close to, the nanomolar range (a KD-value of 10×10⁻⁹ M). Affinity can be measured by methods known in the art, such as, for example, surface plasmon resonance.

The antibodies and nucleic acids according to the disclosure may be generated by methods known by a person skilled in the art. Ausubel et al., Current protocols in Molecular Biology, 5^{th} edition, John Wiley and sons (2011) provides details on cloning and protein expression, relevant parts of which is hereby incorporated by reference.

Antibodies according to the invention are conveniently produced by expressing the nucleic acid encoding it, for example in a cell system. This enables the production of the antibodies in large amounts. Systems for cloning and expression of a protein are well known. Suitable hosts include bacteria (such as E. coli) yeast, baculovirus and eukaryotic cells such as HeLa cells, Chinese hamster ovary cells (CHO cells) and others. Expression may conveniently be achieved by culturing the host containing the nucleic acid under appropriate conditions. The antibodies may then be isolated and purified using methods known to a person skilled in the art.

As described above, the antibody may comprise or consist of fragments of antibodies, homologues to antibodies, chimeric antibodies, fusion proteins, and other functional equivalents. The antibody may have at least one human constant region. The at least one human constant region may be the constant regions of human IgG, in particular human IgG1 or human IgG4. The antibody may comprise a human antibody framework, such that the CDRs according to the invention may substitute the CDRs of an antibody, for example a whole human antibody.

The antibody that carries the CDRs of the invention may generally comprise one antibody heavy chain sequence and one light chain sequence or substantial portions thereof in which the CDR1, CDR2 and CDR3 regions are located at locations corresponding to the CDR1, CDR2 and CDR3 regions of naturally-occurring VH and VL antibody variable domains encoded by rearranged immunoglobin genes. Thus, the amino acid sequences SEQ ID NO 19, 25, 31 and 71 will replace the CDR1 of a heavy chain, the amino acid sequences SEQ ID NO 1, 7, 13 and 70 will replace the CDR1 of a light chain, the amino acid sequences SEQ ID NO 20, 26, 32 and 79 will replace the CDR2 of a heavy chain, the amino acid sequences SEQ ID NO 2, 8, 14 and 78 will replace the CDR2 of a light chain, the amino acid sequences 21, 27, 33 and 88 will replace the CDR3 of a heavy chain; and the amino acid sequences SEQ ID NO 3, 9, 15 and 87 will replace the CDR3 of a light chain.

The framework regions of the variable regions may be derived from any germline or rearranged variable domain, or may be a synthetic variable domain based on consensus sequences of known human variable domains. The CDR sequences of the invention may be introduced into a repertoire of variable domains lacking CDR sequences using recombinant DNA technology. Methods for this are known, for example from Marks et al., Bio/Technology 10:779-783, 1992.

Examples of suitable framework regions are those regions encoded by the nucleic acid sequences presented in the attached sequence listing, where SEQ ID NO 43-45, 49-51, 55-57, and 65, 73, and 81 encode framework regions for heavy chains and SEQ ID NO 40-42, 46-48, 52-54, and 64, 72, and 80 encode framework regions for light chains. A suitable set of framework regions can also be obtained by translating one of the disclosed framework region sequences and then removing the CDR sequences. This can be carried out by aligning the resulting peptide sequence with the CDR sequences using, for example Blast2-sequences. Further useful combinations of framework regions and CDRs can be identified by experimentation.

The antibodies disclosed herein may comprise or consist of the heavy chain sequences and the right chain sequences, including CDR sequences, encoded by the nucleic acids of Table 5.

The antibodies and nucleic acids according the invention are preferably isolated.

A nucleic acid that encodes an antibody according to the invention forms a separate aspect of the Invention. Examples of such nucleic acids can be found in the sequences disclosed in Table 5 which encodes CDRs as well as framework regions.

TABLE 5
Coding sequences

| | Heavy Chain | |
|---|---|---|
| | CDR1 sequence | |
| Antibody | SEQ ID NO | Light Chain CDR1 Sequence SEQ ID NO |
| ACPA1 | 58 | 61 |
| ACPA10 | 59 | 62 |
| ACPA11 | 60 | 63 |
| ACPA42 | 69 | 68 |

The nucleic acid sequences disclosed herein may vary to some extent as many different DNA- or RNA sequences can encode the same peptide. Nucleic acids may be generated by molecular biology methods known to a person skilled in the art. The sequences of the nucleic acid can be easily obtained by reverse-transcribing the peptide sequences disclosed herein using appropriate software.

Such software is well known to the skilled person and can be found for example at www.expasy.org. The sequences may then also be codon-optimized for the expression system used in the particular case (e.g. bacteria, yeast baculovirus, HeLa). Conveniently the nucleic acids are generated by synthesis and cloned into a suitable expression plasmid. Such a plasmid usually contains promoter sequences, secretion sequences, polyadenylation sequences, genes for selection, origins of replication and other elements known in the art.

A further aspect provides a host cell containing a nucleic acid as disclosed herein. The host cell may be a HEK293 cell.

The antibodies and nucleic acids disclosed herein may also be wholly or partly generated by chemical synthesis.

In one embodiment the antibody is a human antibody. When using the antibodies as a research tool in living animals they are conveniently such that they do not cause immunity in that animal. Thus, when testing in mice any constant regions of the antibody are preferably of murine origin.

Each of the following antibodies are encompassed by the disclosure and can be freely combined with other features of the invention:

An antibody (ACPA1) wherein the heavy chain CDR1 is SEQ ID NO 19, the light chain CDR1 is SEQ ID NO 1, the heavy chain CDR2 is SEQ ID NO 20, the light chain CDR2 is SEQ ID NO 2, the heavy chain CDR3 is SEQ ID NO 21 and the light chain CDR3 is SEQ ID NO 3.

An antibody (ACPA10) wherein the heavy chain CDR1 is SEQ ID NO 25, the light chain CDR1 is SEQ ID NO 7, the heavy chain CDR2 is SEQ ID NO 26, the light chain CDR2 is SEQ ID NO 8, the heavy chain CDR3 is SEQ ID NO 27 and the light chain CDR3 is SEQ ID NO 9.

An antibody (ACPA11) wherein the heavy chain CDR1 is SEQ ID NO 31, the light chain CDR1 is SEQ ID NO 13, the heavy chain CDR2 is SEQ ID NO 32, the light chain CDR2 is SEQ ID NO 14, the heavy chain CDR3 is SEQ ID NO 33 and the light chain CDR3 is SEQ ID NO 15.

An antibody (ACPA42) wherein the heavy chain CDR1 is SEQ ID NO 71, the light chain CDR1 is SEQ ID NO 70, the heavy chain CDR2 is SEQ ID NO 79, the light chain CDR2 is SEQ ID NO 78, the heavy chain CDR3 is SEQ ID NO 88 and the light chain CDR3 is SEQ ID NO 87.

Another aspect is an antibody according to the invention for use in the prevention, treatment and/or alleviation of rheumatoid arthritis.

Preferably such an antibody is a dominant negative antibody, for example an antibody that is modified such that it does not trigger a complement activation or activation of other effector mechanisms that are dependent on the glycosylation of the Fc and/or Fab parts of the antibody. Such an antibody will compete with the pathogenic antibodies of the patient for binding to the epitope, but it will not trigger complement.

Before treatment commences, it should be established that the disease of the patient is caused by antibodies that bind to the same epitopes as those of the Invention (at least one of cit-vim and cit-fib). This can be carried out with ELISA using serum from the patient. The patient is suitably treated with an antibody that binds to the same epitope as the pathologic antibody. This can be analyzed with the diagnostic method for treatment set out below.

“Dominant negative” antibodies are antibodies that compete with the disease-causing antibody for binding to its epitope, but lack the ability to trigger the disease-causing mechanism. The disease causing mechanism can be inflammation, complement activation or binding to Fc receptors of effector cells such as macrophages. An antibody can be made dominant negative by modifying the antibody. This can be carried out, for example, by modification of the glycosylation of the Fc or Fab parts of the antibodies, so that complement inducing molecules (for example the Fc-receptor) cannot bind to the antibody. Such modifications can be achieved by several different procedures including modification of the glycosylation during the production of monoclonal antibodies in in vitro systems, or by means of cleavage of certain sugars in the Fc or Fab parts of an immunoglobulin by enzymes, including treatment of the antibodies in vitro with the bacterial-derived endoS enzyme (Allhorn et al, Blood. 2010 June 17; 115(24): 5080-5088). Alternatively, glycosylation sites in the antibody can be removed by modifying the DNA encoding the antibody using molecular biology techniques.

Yet another aspect of the invention is a method of treating rheumatoid arthritis comprising administrating to a patient in need there of an antibody according to the Invention. The method for treatment may comprise the step of, prior to administering the antibody to the patient, selecting the antibody to be administered to the patient. Suitably this is carried out by analyzing the nature of the autoimmune reaction in the patient. The method can comprise the steps of:

1) providing a sample comprising antibodies from the patient;
2) testing the binding of antibodies in the sample towards at least one epitope selected from the group consisting of SEQ ID NO 37, SEQ ID NO 38 and SEQ ID NO 39;
3) administering an antibody to the patient.

Testing can be carried out using, for example, an ELISA method where the peptide is immobilized. The sample comprising antibodies can be isolated from the patient, for example isolated from synovial fluid or plasma.

When used in the treatment of a human, the antibody is preferably mainly of human origin, as to not cause the production of antibodies against the antibodies.

For therapeutic use, the antibody suitably is stable after administrated to a human patient. For example, it should have a long half-life in humans and not be broken down by proteases short time after administration. Suitable, the antibody has a half-life of weeks rather than days.

Administration to a human patient may be done in a variety of ways, including but not limited to orally, subcutaneously, intravenously, and parenterally. In one aspect of the Invention administration is carried out intravenously. For therapeutic use, the antibody is suitable formulated together with pharmaceutically acceptable buffers, preservatives, carriers and other excipients known to a person skilled in the art. Wang et al, Journal of Pharmaceutical Sciences, Volume 96, issue 1, pages 1-26, January 2007 describes formulations of antibodies. The antibodies are preferably administered in an effective amount that minimizes any side effects. The dosage can be in the range of from 1 to 50 mg/kg of patient body weight. The appropriate dose can be determined by methods known in the art and by persons skilled in the art, such as the treating physician.

The antibodies disclosed herein can be used in diagnosis or as a research tool. For example, one or more antibodies disclosed herein may be included as positive controls in a diagnostic kit for testing for the presence of autoantibodies with reactivity against rheumatoid arthritis-specific antigens, in particular citrullinated enolase, citrullinated vimentin, citrullinated fibrinogen and/or collagen type II.

Preferred concentrations for the antibodies when used in vitro can be from 10 ng/ml to 50 μg/ml. The appropriate concentration which yields a suitable signal with low background (good signal to noise ratio) can be found by a person skilled in the art, such as the treating physician. Suitable medium for the dilution of the antibodies are also known in the art and can, for example, be phosphate buffered saline optionally with a supplement of BSA.

One further aspect is a diagnostic kit or a prognostic kit that comprises an antibody according to the Invention. Such a kit preferably comprises an ELISA plate or other platform for antibody analysis as well as reagents for detection of antibodies, such as labeled-anti-human antibodies and suitable buffers. Thus the antibodies according to the invention can be used for in vitro diagnosis.

A further aspect comprises an antibody according to the invention for use in diagnosis of a disease, preferably rheumatoid arthritis, and the use of an antibody according to the Invention for the manufacture of a diagnostic.

Another aspect relates to the use of an antibody as defined above for the manufacture of a medicament for the treatment of rheumatoid arthritis.

### EXAMPLES

### Example 1

In order to identify autoantibodies in RA, antibody-coding genes were cloned from individual antibody secreting cells of patients with RA. Immunoglobulin genes were cloned and the cognate antibodies were expressed from the individual cells. This allows the identification of actual pairs of heavy chains and light chains in naturally occurring antibodies.

Briefly, antibody secreting cells were isolated from consenting RA-patients and cDNA was generated from the individual B-cells. Variably heavy- and light chain transcripts were amplified from each isolated individual cell using specific primers, and thereafter sequenced. The variable regions of the heavy chains and the light chains have the DNA sequences shown in table 5 and the translated and analyzed CDR regions the sequences as shown in Tables 2-4.

### Example 2

Coding regions from example 1 above, were separately cloned into expression vectors in frame with the gene for the constant region of heavy chain or light chain of human, as appropriate. The expression was under control of the human cytomegalovrus (HCMV) promoter. An appropriate cell-line was co-transfected with paired expression plasmids (one encoding the variable light chain and one encoding the variable heavy chain). Expressed and purified antibodies were tested for reactivity against the following RA-associated antigens: citrullinated CEP-1, citrullinated fibrinogen peptide, and citrullinated vimentin peptide (Table 6).

TABLE 6
RA-associated antigens

| Antigen | Peptide sequence | SEQ ID NO |
|---|---|---|
| CEP-1 | CKIHAXEIFDSXGNPTVEC | 37 |
| Vim60-75 | VYATXSSAVXLXSSVP | 38 |
| Fib36-52 | NEEGFFSAXGHRPLDKK | 39 |

X = citrulline

The reactivity of the isolated antibodies is shown in Table 7 where the arbitrary units (Au)/ml values relates to a standard, which is a serum pool consisting of polyclonal citruline reactive antibodies.

TABLE 7
Reactivity of isolated antibodies

| | | | | | cit-vim |
|---|---|---|---|---|---|
| | | | | | (Au/ml |
| | | | cit-fib (Au/ml | | at |
| Antibody | CEP-1 | cit-fib | at 5 ug/ml) | cit-vim | 5 ug/ml)* |
| ACPA1 | − | + | 14 | ++ | 60 |
| ACPA10 | − | − | − | +++++ | 496 |
| ACPA11 | − | + | 21 | − | − |
| ACPA42 | − | ++++ | 50* | − | − |

*Au/ml at 1.3 ug/ml

### Example 3

### Isolation of Antibodies

Synovial fluid samples were obtained from patients suffering from rheumatoid arthritis. Antibodies were isolated from said samples using the fluorescent foci method essentially as described in Lightwood et al., J Mol Biol, 2013, but without an initial B cell culturing step, incorporated herein by reference.

### Example 4

### SPR Binding Data for Human Anti-Citruline Antibodies

### Method

Surface plasmon resonance (SPR) was performed using a Blacore T200 (GE Healthcare). All experiments were performed at 25□C. Streptavidin was immobilised on a CM5 Sensor Chip (GE Healthcare) via amine coupling chemistry to a capture level of ˜1000 response units. HBS-EP+ buffer (10 mM HEPES pH 7.4, 150 mM NaCl, 3 mM EDTA, 0.05% (v/v) surfactant P20, (GE Healthcare) was used as the running buffer. For each assay, a 20□I injection (10□l/min) of 100 nM biotinylated peptide (fibrinogen 36-52, control peptide, enolase-1 peptide, or vimentin 60-75), was used to achieve approximately 100 RU of Irreversibly captured citrullinated or argine control peptide. A 3 minute injection of anti-citrullinated antibodies at 33 nM, was passed over the immobilized peptide (10 minute dissociation) at a flow rate of 30□l/min in running buffer. The surface was regenerated at a flow-rate of 10□l/min by a 3 minute injection of 4M MgCl2. Double referenced background subtracted binding curves were analyzed using the T200 Evaluation software (version 1.0) following standard procedures. Kinetic parameters were roughly approximated for each 33 nM IgG injection using a bivalent fitting algorithm.

### Results

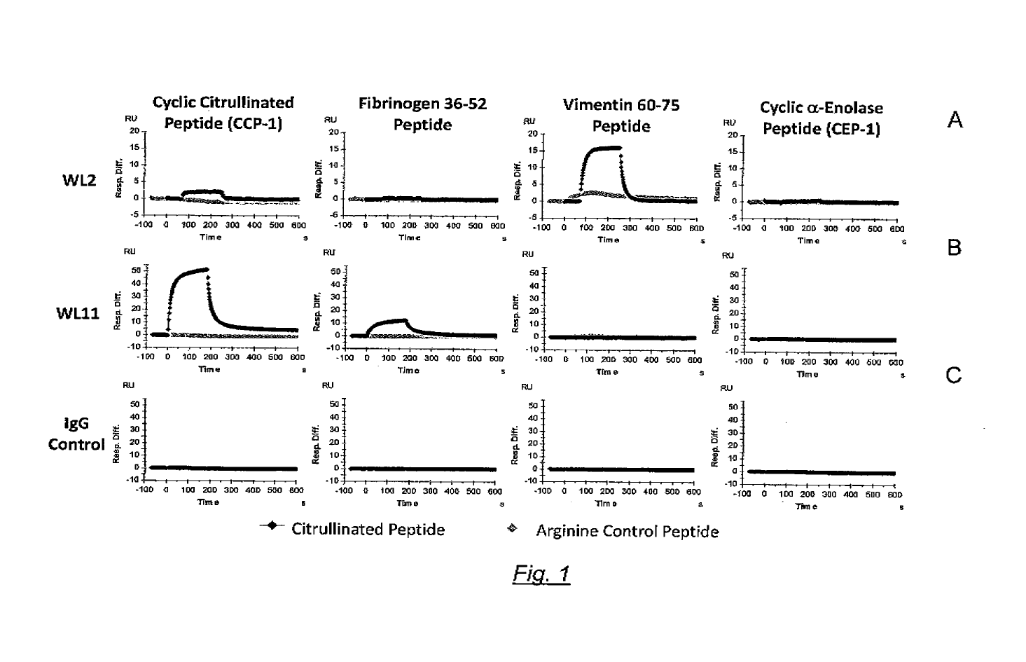

Antibodies ACPA1, ACPA11 and control IgG were tested for their ability to bind immobilized arginine and citrulline containing peptides using SPR according to the method described previously. Antibody ACPA1 was shown to bind specifically to the cyclic citruline containing peptide (CCP-1) and also to the vimentin containing citrulline peptide but only very weakly to the fibrinogen and not at all to the alpha-enolase citrulline peptides (FIG. 1a). ACPA1 does not bind any of the arginine containing peptides. Thus ACPA1 may prove a useful tool to specifically recognize vimentin associated with citruline modification as part of an on-going autoimmune or Inflammatory reaction.

Antibody ACPA11 was shown to bind specifically to the cyclical citrulline containing peptide (CCP-1) and also to the fibrinogen containing citruline peptide but not to the enolase or vimentin citrulline peptides (FIG. 1b). ACPA11 does not bind any of the arginine containing peptides. Thus ACPA11 may prove a useful tool to specifically recognize fibrinogen associated with citrulline modification as part of an on-going autoimmune or Inflammatory reaction. As can be seen in FIG. 1c the control IgG did not bind any of the arginine or citruline containing CCP-1, fibrinogen (Fib), enolase-1 (Eno) or vimentin (Vim) containing peptides immobilized to the chip surface.

### REFERENCES TO METHODS

The experimental part of the present disclosure was performed using methods described in the below references, unless otherwise indicated:
Kinloch, A., V. Tatzer, R. Wait, D. Peston, K. Lundberg, P. Donatien, D. Moyes, P. C. Taylor, and P. J. Venables. 2005. Identification of citrullinated alpha-enolase as a candidate autoantigen in rheumatoid arthritis. Arthritis Res Ther 7:R1421-1429
Lightwood, D. et al., 2013, The Discovery, Engineering and Characterisation of a Highly Potent Anti-Human IL-13 Fab Fragment Designed for Administration by Inhalation, J Mol Biol 425(3):577-93
Lundberg, K., A. Kinloch, B A. Fisher, N. Wegner, R. Wait, P. Charles, T. R. Mikuls, and P. J. Venables. 2008. Antibodies to citrullinated alpha-enolase peptide 1 are specific for rheumatoid arthritis and cross-react with bacterial enolase. Arthritis and rheumatism 58:3009-3019
Snir, O., M. Rieck, J. A. Gebe, B. B. Yue, C. A Rawlings, G. Nepom, V. Malmstrom, and J. H. Buckner. 2011. Identification and functional characterization of T cells reactive to citrullinated vimentin in HLA-DRB1*0401-positive humanized mice and rheumatoid arthritis patients. Arthritis and rheumatism 63:2873-2883
Verpoort, K. N., K. Cheung, A. Ioan-Facsinay, A. H. van der Helm-van MII, J. K. de Vrles-Bouwstra, C. F. Alaart, J. W. Drifhout, R. R. de Vrles, F. C. Breedveld, T. W. Huizinga, G. J. Pruijn, and R. E. Toes. 2007. Fine specificity of the anti-citrullinated protein antibody response is influenced by the shared epitope alleles. Arthritis and rheumatism 56:3949-3952

1. An antibody that binds to at least one citrullinated epitope, said antibody comprising a heavy chain CDR1 (HCDR1); a heavy chain CDR2 (HCDR2); and a heavy chain CDR3 (HCDR3) selected from the following combinations of amino acid sequences:
Combination No.

| | 1 | 2 | 3 | 4 |
|---|---|---|---|---|
| HCDR1, SEQ ID No. | 19 | 25 | 31 | 69 |
| HCDR2, SEQ ID No. | 20 | 26 | 32 | 79 |
| HCDR3, SEQ ID No. | 21 | 27 | 33 | 88 |

or a substantially identical antibody thereof.

2. The antibody according to claim 1, comprising a heavy chain CDR1 (HCDR1); a light chain CDR1 (LCDR1); a heavy chain CDR2 (HCDR2); a light chain CDR2 (LCDR2); a heavy chain CDR3 (HCDR3); and a light chain CDR3 (LCDR3) selected from the following combinations of sequences:
Combination No.

| | 1 | 2 | 3 | 4 |
|---|---|---|---|---|
| HCDR1, SEQ ID No. | 19 | 25 | 31 | 69 |
| LCDR1, SEQ ID No. | 1 | 7 | 13 | 68 |
| HCDR2, SEQ ID No. | 20 | 26 | 32 | 79 |
| LCDR2, SEQ ID No. | 2 | 8 | 14 | 78 |
| HCDR3, SEQ ID No. | 21 | 27 | 33 | 88 |
| LCDR3, SEQ ID No. | 3 | 9 | 15 | 87 |

or a substantially identical antibody thereof.

3. The antibody according to claim 1, wherein said antibody binds to at least one citrullinated epitope selected from the group consisting of cit-vim (SEQ ID NO 38) and cit-fib (SEQ ID NO 39).

4. The antibody according to claim 1, wherein the heavy chain CDR1 is SEQ ID NO 19, the light chain CDR1 is SEQ ID NO 1, the heavy chain CDR2 is SEQ ID NO 20, the light chain CDR2 is SEQ ID NO 2, the heavy chain CDR3 is SEQ ID NO 21, and the light chain CDR3 is SEQ ID NO 3.

5. The antibody according to claim 1, wherein the heavy chain CDR1 is SEQ ID NO 25, the light chain CDR1 is SEQ ID NO 7, the heavy chain CDR2 is SEQ ID NO 26, the light chain CDR2 is SEQ ID NO 8, the heavy chain CDR3 is SEQ ID NO 27 and the light chain CDR3 is SEQ ID NO 9.

6. The antibody according to claim 1, wherein the heavy chain CDR1 is SEQ ID NO 31, the light chain CDR1 is SEQ ID NO 13, the heavy chain CDR2 is SEQ ID NO 32, the light chain CDR2 is SEQ ID NO 14, the heavy chain CDR3 is SEQ ID NO 33 and the light chain CDR3 is SEQ ID NO 15.

7. The antibody according to claim 1, wherein the heavy chain CDR1 is SEQ ID NO 71, the light chain CDR1 is SEQ ID NO 70, the heavy chain CDR2 is SEQ ID NO 79, the light chain CDR2 is SEQ ID NO 78, the heavy chain CDR3 is SEQ ID NO 88 and the light chain CDR3 is SEQ ID NO 87.

8. The antibody according to claim 1, comprising at least one human constant region.

9. The antibody according to claim 1, comprising at least one human constant region of human IgG.

10. The antibody according to claim 1, comprising at least one human constant region of human IgG1.

11. The antibody according to claim 1, least one human constant region of human IgG4.

12. The antibody according to claim 1, wherein said antibody is modified with respect to its carbohydrate structure or protein backbone.

13. A nucleic acid encoding an antibody that binds to at least one citrullinated epitope, said antibody comprising a heavy chain CDR1 (HCDR1): a heavy chain CDR2 (HCDR2); and a heavy chain CDR3 (HCDR3) selected from the following combinations of amino acid sequences:
Combination No.

| | 1 | 2 | 3 | 4 |
|---|---|---|---|---|
| HCDR1, SEQ ID No. | 19 | 25 | 31 | 69 |
| HCDR2, SEQ ID No. | 20 | 26 | 32 | 79 |
| HCDR3, SEQ ID No. | 21 | 27 | 33 | 88 |

or a substantially identical antibody thereof.

14. The nucleic acid according to claim 13 comprising a sequence selected from the group consisting of SEQ ID NO 58, SEQ ID NO 59, SEQ ID NO 60, SEQ ID NO 61, SEQ ID NO 62, and SEQ ID NO 63.

15. An antibody that binds to at least one citrullinated epitope, said antibody comprising a heavy chain CDR1 (HCDR1): a heavy chain CDR2 (HCDR2); and a heavy chain CDR3 (HCDR3) selected from the following combinations of amino acid sequences:
Combination No.

| | 1 | 2 | 3 | 4 |
|---|---|---|---|---|
| HCDR1, SEQ ID No. | 19 | 25 | 31 | 69 |
| HCDR2, SEQ ID No. | 20 | 26 | 32 | 79 |
| HCDR3, SEQ ID No. | 21 | 27 | 33 | 88 |

or a substantially identical antibody thereof for use in the treatment of rheumatoid arthritis.

16. The antibody for use according to claim 15 which is a dominant negative antibody.

17. A method of treating rheumatoid arthritis comprising administrating to a patient in need thereof a therapeutically effective amount of an antibody that binds to at least one citrullinated epitope, said antibody comprising a heavy chain CDR1 (HCDR1); a heavy chain CDR2 (HCDR2); and a heavy chain CDR3 (HCDR3) selected from the following combinations of amino acid sequences:
Combination No.

| | 1 | 2 | 3 | 4 |
|---|---|---|---|---|
| HCDR1, SEQ ID No. | 19 | 25 | 31 | 69 |
| HCDR2, SEQ ID No. | 20 | 26 | 32 | 79 |
| HCDR3, SEQ ID No. | 21 | 27 | 33 | 88 |

or a substantially identical antibody thereof.

18. The method according to claim 17, wherein the antibody is a dominant negative antibody.

19. A diagnostic kit or prognostic kit comprising an antibody according to claim 1.

20. An antibody according to claim 1 for use in diagnosis.

21. An antibody according to claim 1, for use in diagnosis of rheumatoid arthritis or a step in such diagnosis.

22. An antibody according to claim 1, for use in diagnosis, wherein the diagnosis includes a prediction of risk of developing rheumatoid arthritis and/or diagnosis of rheumatoid arthritis at an early stage, before the onset of clinical symptoms.


SEQUENCE LISTING (88 sequences)

SEQ ID NO: 1 - protein, 11 aa, Homo sapiens
  PEPTIDE (1)..(11): ACPA1L LCDR1
RASEDIGSLLA

SEQ ID NO: 2 - protein, 7 aa, Homo sapiens
  PEPTIDE (1)..(7): ACPA1L LCDR2
KASSLES

SEQ ID NO: 3 - protein, 9 aa, Homo sapiens
  PEPTIDE (1)..(9): ACPA1L LCDR3
QQYNGPSET

SEQ ID NO: 4 - DNA, 33 bp, Homo sapiens
  misc_feature (1)..(33): ACPA1L LCDR1
cgggccagtgaggatatcggtagtttgttggcc

SEQ ID NO: 5 - DNA, 21 bp, Homo sapiens
  misc_feature (1)..(21): ACPA1L LCDR2
aaggcgtccagtttagaaagt

SEQ ID NO: 6 - DNA, 27 bp, Homo sapiens
  misc_feature (1)..(27): ACPA1L LCDR3
caacaatacaatggtccctcggagacg

SEQ ID NO: 7 - protein, 11 aa, Homo sapiens
  PEPTIDE (1)..(11): ACPA10 LCDR1
GGDDIENQNVN

SEQ ID NO: 8 - protein, 7 aa, Homo sapiens
  PEPTIDE (1)..(7): ACPA10 LCDR2
FDTRRPS

SEQ ID NO: 9 - protein, 11 aa, Homo sapiens
  PEPTIDE (1)..(11): ACPA10 LCDR3
QVYDRKTDHQV

SEQ ID NO: 10 - DNA, 33 bp, Homo sapiens
  misc_feature (1)..(33): ACPA10 LCDR1
ggtggagacgacattgaaaatcaaaatgtcaac

SEQ ID NO: 11 - DNA, 21 bp, Homo sapiens
  misc_feature (1)..(21): ACPA10 LCDR2
tttgataccagacggccctca

SEQ ID NO: 12 - DNA, 33 bp, Homo sapiens
  misc_feature (1)..(33): ACPA10 LCDR3
caggtgtatgataggaagactgatcaccaagtc

SEQ ID NO: 13 - protein, 13 aa, Homo sapiens
  PEPTIDE (1)..(13): ACPA11 LCDR1
SGNTSNIGYNIVN

SEQ ID NO: 14 - protein, 7 aa, Homo sapiens
  PEPTIDE (1)..(7): ACPA11 LCDR2
DNDKRPS

SEQ ID NO: 15 - protein, 11 aa, Homo sapiens
  PEPTIDE (1)..(11): ACPA11 LCDR3
AVWDDDLSGVV

SEQ ID NO: 16 - DNA, 39 bp, Homo sapiens
  misc_feature (1)..(39): ACPA11 LCDR1
tctggaaacacctccaacatcggatataatattgtgaat

SEQ ID NO: 17 - DNA, 21 bp, Homo sapiens
  misc_feature (1)..(21): ACPA11 LCDR2
gataatgataaacggccctcc

SEQ ID NO: 18 - DNA, 33 bp, Homo sapiens
  misc_feature (1)..(33): ACPA11 LCDR3
gcggtgtgggatgacgacctgagtggcgtggtg

SEQ ID NO: 19 - protein, 5 aa, Homo sapiens
  PEPTIDE (1)..(5): ACPA1 HCDR1
GYFIH

SEQ ID NO: 20 - protein, 16 aa, Homo sapiens
  PEPTIDE (1)..(16): ACAP1 HCDR2
WIHPTGDTKYAQKFQG

SEQ ID NO: 21 - protein, 8 aa, Homo sapiens
  PEPTIDE (1)..(8): ACAP1 HCDR3
TNFSPFRH

SEQ ID NO: 22 - DNA, 15 bp, Homo sapiens
  misc_feature (1)..(15): ACAP1 HCDR1
ggctactttatccac

SEQ ID NO: 23 - DNA, 48 bp, Homo sapiens
  misc_feature (1)..(48): ACAP1 HCDR2
tggatccaccctactggagacacaaagtatgcacagaaatttcagggc

SEQ ID NO: 24 - DNA, 24 bp, Homo sapiens
  misc_feature (1)..(24): ACAP1 HCDR3
accaacttcagcccattccgacac

SEQ ID NO: 25 - protein, 5 aa, Homo sapiens
  PEPTIDE (1)..(5): ACAP10 HCDR1
GYFIH

SEQ ID NO: 26 - protein, 16 aa, Homo sapiens
  PEPTIDE (1)..(16): ACAP10 HCDR2
WIHPTGDTKYAQKFQG

SEQ ID NO: 27 - protein, 8 aa, Homo sapiens
  PEPTIDE (1)..(8): ACAP10 HCDR3
TNFSPFRH

SEQ ID NO: 28 - DNA, 21 bp, Homo sapiens
  misc_feature (1)..(21): ACAP10 HCDR1
gataccacttactactggggc

SEQ ID NO: 29 - DNA, 48 bp, Homo sapiens
  misc_feature (1)..(48): ACAP10 HCDR2
agtatctattaccgggggaacacccactacaattcgtccctgaggagt

SEQ ID NO: 30 - DNA, 18 bp, Homo sapiens
  misc_feature (1)..(18): ACAP10 HCDR3
ctcgacccatttgactac

SEQ ID NO: 31 - protein, 6 aa, Homo sapiens
  PEPTIDE (1)..(6): ACAP11 HCDR1
NDKFWG

SEQ ID NO: 32 - protein, 16 aa, Homo sapiens
  PEPTIDE (1)..(16): ACAP11 HCDR2
SINPLGRTFYTTSLKD

SEQ ID NO: 33 - protein, 9 aa, Homo sapiens
  PEPTIDE (1)..(9): ACAP11 HCDR3
DGEGVLFDE

SEQ ID NO: 34 - DNA, 21 bp, Homo sapiens
  misc_feature (1)..(21): ACAP11 HCDR1
agaaatgataaattctggggc

SEQ ID NO: 35 - DNA, 48 bp, Homo sapiens
  misc_feature (1)..(48): ACAP11 HCDR2
agtatcaatccccttggcagaaccttctacaccacgtccctcaaggat

SEQ ID NO: 36 - DNA, 27 bp, Homo sapiens
  misc_feature (1)..(27): ACAP11 HCR3
gacggagagggagtcctctttgacgag

SEQ ID NO: 37 - protein, 19 aa, Human
  MISC_FEATURE (1)..(19): CEP-1
CKIHAXEIFDSXGNPTVEC

SEQ ID NO: 38 - protein, 16 aa, Homo sapiens
  MISC_FEATURE (1)..(16): Vim60-75
VYATXSSAVXLXSSVP

SEQ ID NO: 39 - protein, 17 aa, Homo sapiens
  MISC_FEATURE (1)..(17): Fib36-52
NEEGFFSAXGHRPLDKK

SEQ ID NO: 40 - DNA, 69 bp, Homo sapiens
  misc_feature (1)..(69): L frame work region
gacatccagatgacccagtctccagccaccctgtctgcatctgttggagacagagtcaccatcacttgc

SEQ ID NO: 41 - DNA, 45 bp, Homo sapiens
  misc_feature (1)..(45): L frame work region
tggttgcagcagaaaccagggaaagcccctaaactcctgattcat

SEQ ID NO: 42 - DNA, 96 bp, Homo sapiens
  misc_feature (1)..(96): L frame work region
ggggtcccatcaagattcagcggcagtggatctgagacagaatttactctcaccattagcagcctgcagcctgatgattttgcaacttactactgt

SEQ ID NO: 43 - DNA, 90 bp, Homo sapiens
  misc_feature (1)..(90): H framework region
caggtgcagctggtgcagtctggggctgaggtgcagaagcctggggcctcagtgaaggtctcctgcaaggcttctggatacacgttcacc

SEQ ID NO: 44 - DNA, 42 bp, Homo sapiens
  misc_feature (1)..(42): H framework region
tgggtgcgacaggcccctgggcagggtctggagtggatgggg

SEQ ID NO: 45 - DNA, 96 bp, Homo sapiens
  misc_feature (1)..(96): H framework region
agggtcaatatgaccagtgacgcgtccattagcacagcctacctggagttgancagcctgacatctgacgacacggccacatattactgcgcgagg

SEQ ID NO: 46 - DNA, 66 bp, Homo sapiens
  misc_feature (1)..(66): L framework region
tcctatgtcctgactcagccaccctcagtgtcgctggccccgggagagacggccacaattacttgt

SEQ ID NO: 47 - DNA, 45 bp, Homo sapiens
  misc_feature (1)..(45): L framework region
tggtatcagcagaagtcaggtcaggcccctatgctgctcatcttc

SEQ ID NO: 48 - DNA, 96 bp, Homo sapiens
  misc_feature (1)..(96): L framework region
gggatcccggagcgattctctggctccaggtctgaggacacggccaacctgaccatcaccagggtcgaggccggggatgacgccgactatttctgt

SEQ ID NO: 49 - DNA, 90 bp, Homo sapiens
  misc_feature (1)..(90): H framework region
cacctgcacttgcaggagtcgggcccaggacttgtgaagccttcggagaccctgtccctcacctgcactgtctctggtggctccatcaac

SEQ ID NO: 50 - DNA, 42 bp, Homo sapiens
  misc_feature (1)..(42): H framework region
tggattcgccagccccccgggaagggactggagtggattggg

SEQ ID NO: 51 - DNA, 96 bp, Homo sapiens
  misc_feature (1)..(96): H framework region
cgcgtcaccatgtctgtcgacacttccaagaaccgattctccctgaaggtcacttctgtgactgccgcagacacggctgtctattactgtgcgaga

SEQ ID NO: 52 - DNA, 66 bp, Homo sapiens
  misc_feature (1)..(66): L framework region
cagcctgtaatgactcagccaccctcagcgtgtgggacccccgggcagagggtcaccattccttgt

SEQ ID NO: 53 - DNA, 45 bp, Homo sapiens
  misc_feature (1)..(45): L framework region
tggtaccaacaggtcccagggacggcccccaaactcctcatctat

SEQ ID NO: 54 - DNA, 96 bp, Homo sapiens
  misc_feature (1)..(96): H framework region
ggggtccctgaccgattctcagcctccaagtctgtcacctcagcctccctggtcatcagtggcctccagtctgcagatgaggctgactattattgt

SEQ ID NO: 55 - DNA, 84 bp, Homo sapiens
  misc_feature (1)..(84): H framework region
cagctgcaggagtcgggcccaaaactggtgaagacttcggagaccctgtccctcaattgcagtgtctctcgtgagtccatcgtg

SEQ ID NO: 56 - DNA, 42 bp, Homo sapiens
  misc_feature (1)..(42): H framework region
tgggtccgccagccccccgggaagggtcttgaatggattggc

SEQ ID NO: 57 - DNA, 96 bp, Homo sapiens
  misc_feature (1)..(96): H framework region
cgcataagaatttcatttgacacgtccgagggatacttttccctgagtgtcgcctctgtgaccgccgcagacacggccatctattactgtgcgaca

SEQ ID NO: 58 - DNA, 673 bp, Homo sapiens
  misc_feature (1)..(673): Light chain
aagcttcgaagccaccatggacatgagggtccccgctcagtccctggggctcctgctgctctggctcccaggtgccaaatgtgacatccagatgacccagtctccagccaccctgtctgcatctgttggagacagagtcaccatcacttgccgggccagtgaggatatcggtagtttgttggcctggttgcagcagaaaccagggaaagcccctaaactcctgattcataaggcgtccagtttagaaagtggggtcccatcaagattcagcggcagtggatctgagacagaatttactctcaccattagcagcctgcagcctgatgattttgcaacttactactgtcaacaatacaatggtccctcggagacgttcggccaagggaccaaagtggatatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacc

SEQ ID NO: 59 - DNA, 644 bp, Homo sapiens
  misc_feature (1)..(644): light chain
aagcttcgaagccaccatggccagcttccctcccctcctcggcctcctctctcactgcacagactctgtgacctcctatgtcctgactcagccaccctcagtgtcgctggccccgggagagacggccacaattacttgtggtggagacgacattgaaaatcaaaatgtcaactggtatcagcagaagtcaggtcaggcccctatgctgctcatcttctttgataccagacggccctcagggatcccggagcgattctctggctccaggtctgaggacacggccaacctgaccatcaccagggtcgaggccggggatgacgccgactatttctgtcaggtgtatgataggaagactgatcaccaagtcttcggacctgggaccacggtcaccgtcctaggtcagcccaaggctgcacccagtgtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccaca

SEQ ID NO: 60 - DNA, 678 bp, Homo sapiens
  misc_feature (1)..(678): light chain
aagcttcgaagcnaccatggcctggactcctctctngnccctcctcattcactgtgcagggtcctgggcccagcctgtaatgactcagccaccctcagcgtgtgggacccccgggcagagggtcaccattccttgttctggaaacacctccaacatcggatataatattgtgaattggtaccaacaggtcccagggacggcccccaaactcctcatctatgataatgataaacggccctccggggtccctgaccgattctcagcctccaagtctgtcacctcagcctccctggtcatcagtggcctccagtctgcagatgaggctgactattattgtgcggtgtgggatgacgacctgagtggcgtggtgatcggcgaagggaccaagctgaccgtcctcggtcagcccaaggctgcacccagtgtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagg

SEQ ID NO: 61 - DNA, 428 bp, Homo sapiens
  misc_feature (1)..(428): heavy chain
atggactggacctggaggatcctcttcttggtggccgcagccacaggagcccactcccaggtgcagctggtgcagtctggggctgaggtgcagaagcctggggcctcagtgaaggtctcctgcaaggcttctggatacacgttcaccggctactttatccactgggtgcgacaggcccctgggcagggtctggagtggatggggtggatccaccctactggagacacaaagtatgcacagaaatttcagggcagggtcaatatgaccagtgacgcgtccattagcacagcctacctggagttgancagcctgacatctgacgacacggccacatattactgcgcgaggaccaacttcagcccattccgacactggggccgtggcaccctggtcactgtctcgagcgcttctacaaagggcccatcggt

SEQ ID NO: 62 - DNA, 426 bp, Homo sapiens
  misc_feature (1)..(426): heavy chain
atgaaacccctgtggttcttcctcctgctggtggcgactcccagatgggtcctgtgccacctgcacttgcaggagtcgggcccaggacttgtgaagccttcggagaccctgtccctcacctgcactgtctctggtggctccatcaacgataccacttactactggggctggattcgccagccccccgggaagggactggagtggattgggagtatctattaccgggggaacacccactacaattcgtccctgaggagtcgcgtcaccatgtctgtcgacacttccaagaaccgattctccctgaaggtcacttctgtgactgccgcagacacggctgtctattactgtgcgagactcgacccatttgactactggggccgtggcaccctggtcactgtctcgagcgcttctacaaagggcccatcg

SEQ ID NO: 63 - DNA, 426 bp, Homo sapiens
  misc_feature (1)..(426): heavy chain
atgaaacatctgtggttcttcctcctgctggtggcggctcccggatgggtcctgtcccacttgcagctgcaggagtcgggcccaaaactggtgaagacttcggagaccctgtccctcaattgcagtgtctctcgtgagtccatcgtgagaaatgataaattctggggctgggtccgccagccccccgggaagggtcttgaatggattggcagtatcaatccccttggcagaaccttctacaccacgtccctcaaggatcgcataagaatttcatttgacacgtccgagggatacttttccctgagtgtcgcctctgtgaccgccgcagacacggccatctattactgtgcgacagacggagagggagtcctctttgacgagtggagccaaggaaccctggtcaccgtctcgagcgcttctacaaag

SEQ ID NO: 64 - DNA, 66 bp, Homo sapiens
  misc_feature (1)..(66): ACPA42 FWR1 nt Light
cagtccgtgttgacgcagccggcctcagtgtctgcggccccaggacagaaggttaccatctcctgc

SEQ ID NO: 65 - DNA, 90 bp, Homo sapiens
  misc_feature (1)..(90): ACPA42 FWR1 nt Heavy
cagccggagttgcaggagtcgggcccaggactggtgaagccttcggaaaccctgtccctcaagtgcaatgtctctggttactccgtcaac

SEQ ID NO: 66 - protein, 22 aa, Homo sapiens
  MISC_FEATURE (1)..(22): ACPA42 FWR1 aa Light
QSVLTQPASVSAAPGQKVTISC

SEQ ID NO: 67 - protein, 30 aa, Homo sapiens
  MISC_FEATURE (1)..(30): ACPA42 FWR1 aa Heavy
QPELQESGPGLVKPSETLSLKCNVSGYSVN

SEQ ID NO: 68 - DNA, 39 bp, Homo sapiens
  misc_feature (1)..(39): ACPA42 CDR1 nt Light
tctggcagcaactccaacattgagagtaatctcgtctcc

SEQ ID NO: 69 - DNA, 21 bp, Homo sapiens
  misc_feature (1)..(21): nt Heavy ACPA42 CDR1
aatgatctgtcctactggggc

SEQ ID NO: 70 - protein, 13 aa, Homo sapiens
  MISC_FEATURE (1)..(13): ACPA42 CDR1aa Light
SGSNSNIESNLVS

SEQ ID NO: 71 - protein, 7 aa, Homo sapiens
  MISC_FEATURE (1)..(7): ACPA42 CDR1 aa Heavy
NDLSYWG

SEQ ID NO: 72 - DNA, 45 bp, Homo sapiens
  misc_feature (1)..(45): ACPA42 FR2 nt Light
tggttccagcagttcccaggaacggcccccagactcctcatttat

SEQ ID NO: 73 - DNA, 42 bp, Homo sapiens
  misc_feature (1)..(42): ACPA42 FR2 nt Heavy
tggatccgccagccccccgggaggggactggaatggattggt

SEQ ID NO: 74 - protein, 15 aa, Homo sapiens
  MISC_FEATURE (1)..(15): ACPA42 FR2 aa Light
WFQQFPGTAPRLLIY

SEQ ID NO: 75 - protein, 14 aa, Homo sapiens
  MISC_FEATURE (1)..(14): ACPA42 FR2 aa Heavy
WIRQPPGRGLEWIG

SEQ ID NO: 76 - DNA, 21 bp, Homo sapiens
  misc_feature (1)..(21): ACPA42 CDR2 nt Light
gacaataataagcgattttca

SEQ ID NO: 77 - DNA, 48 bp, Homo sapiens
  misc_feature (1)..(48): ACPA42 CDR2 nt Heavy
agtgtccattatagtgggcgcacctactacagcccgtccctcaggagt

SEQ ID NO: 78 - protein, 7 aa, Homo sapiens
  MISC_FEATURE (1)..(7): ACPA42 CDR2 aa Light
DNNKRFS

SEQ ID NO: 79 - protein, 16 aa, Homo sapiens
  MISC_FEATURE (1)..(16): ACPA42 CDR2aa Heavy
SVHYSGRTYYSPSLRS

SEQ ID NO: 80 - DNA, 96 bp, Homo sapiens
  misc_feature (1)..(96): ACPA42 FR3 nt Light
ggaactcctgaccgattctctggctcccagtctggcacgtcggccaccctggccatcaccggagtccaggctggggacgaggccgattattactgc

SEQ ID NO: 81 - DNA, 96 bp, Homo sapiens
  misc_feature (1)..(96): ACPA42 FR3 nt Heavy
cgacttagcatctccatagacacgtccaggaactgtttttctctgaatctgacctctgtgaccgccgcagacacggctgtgtattactgtgcgaaa

SEQ ID NO: 82 - skipped

SEQ ID NO: 83 - protein, 32 aa, Homo sapiens
  MISC_FEATURE (1)..(32): ACPA42 FR3 aa Light
GTPDRFSGSQSGTSATLAITGVQAGDEADYYC

SEQ ID NO: 84 - protein, 32 aa, Homo sapiens
  MISC_FEATURE (1)..(32): ACPA42 FR3 aa Heavy
RLSISIDTSRNCFSLNLTSVTAADTAVYYCAK

SEQ ID NO: 85 - DNA, 36 bp, Homo sapiens
  misc_feature (1)..(36): ACPA42 CDR3 nt Light
ggaacatgggattccagtctgagtgctgggctgttc

SEQ ID NO: 86 - DNA, 45 bp, Homo sapiens
  misc_feature (1)..(45): ACPA42 CDR3 nt Heavy
cttggttgcagtggtgggggctgctacttcgactttgactactgg

SEQ ID NO: 87 - protein, 12 aa, Homo sapiens
  MISC_FEATURE (1)..(12): ACPA42 CDR3 aa Light
GTWDSSLSAGLF

SEQ ID NO: 88 - protein, 15 aa, Homo sapiens
  MISC_FEATURE (1)..(15): ACPA42 CDR3 aa Heavy
LGCSGGGCYFDFDYW